(19) United States
(12) Patent Application Publication
(10) Pub. No.: US 2023/0089217 A1
(43) Pub. Date: Mar. 23, 2023

(54) CONFIGURABLE DIAGNOSTIC PLATFORM SYSTEMS AND METHODS FOR PERFORMING CHEMICAL TEST ASSAYS

(71) Applicant: PEEK TECHNOLOGIES, INC., Santa Clara, CA (US)

(72) Inventors: Wilma MANGAN, Santa Clara, CA (US); Tai NGO, Santa Clara, CA (US); Alton B. OTIS, Jr., Santa Clara, CA (US)

(21) Appl. No.: 17/798,846

(22) PCT Filed: Feb. 18, 2021
(86) PCT No.: PCT/US2021/018617

§ 371 Date: Aug. 10, 2022

Related U.S. Application Data
(60) Provisional application No. 62/978,657, filed on Feb. 19, 2020.

(51) Int. Cl. G01N 21/78; B01L 3/00

(52) U.S. Cl. CPC G01N 21/78; B01L 3/502715; G01N 2021/7759

(57) ABSTRACT

Provided are methods and systems that relate to configuring a handheld analyzer. The handheld analyzer may receive an identifier from a dual design test cartridge. The parameter module disposed within the handheld analyzer may determine parameters corresponding to the received test cartridge identifier. The parameter module may then configure the handheld analyzer to perform a test with the dual designed test cartridge using the determined parameters. In an embodiment, the diagnostic test module may determine the test corresponding to the received cartridge identifier and the diagnostic test module configures the handheld analyzer to perform the determined test with the test cartridge. In another embodiment, the dual test cartridge comprises an outer enclosure and an inner enclosure. The outer enclosure comprises a parameter module capable of storing test cartridge identification and may be reused to store new test identifications.

### CROSS-REFERENCE

This application is the U.S. National Stage entry of International Application No. PCT/US2021/018617, filed Feb. 18, 2021, which claims the benefit of U.S. Provisional Application No. 62/978,657, filed Feb. 19, 2020, each of which is entirely incorporated herein by reference.

### BACKGROUND

The present invention relates to an analytical platform system and more particularly to configurable systems for performing chemical test assays.

### SUMMARY

Qualitative and quantitative immuno- and chemical assays and devices and systems for implementing such assays, such as those provided herein, may be used as important tools in a variety of industries, including in the medical and industrial industries. These systems, devices, and methods (assays) may be used for a number of purposes, such as, by way of non-limiting example, the diagnosis of disease conditions, detection of analytes, and for the detection of microbes, such as bacteria.

Often, diagnostic assays are performed in laboratory settings, and involve the use of sophisticated and expensive equipment, that may require specially trained personnel for their operation. Further, assay results may sometimes be unavailable for days or weeks after the samples have been obtained. Thus, presently available diagnostic assays are often costly, time consuming, and not convenient. By contrast, in some instances, provided herein are systems, devices, assays, and component parts thereof that provide cost effective, rapid, versatile, convenient, and/or other beneficial results.

Attempts have been made to develop less costly assays. For example, in some instances, a home self-test for detecting blood components requires the patient to prick a finger with a sterilized lancet, apply a drop of blood sample to a sample application area on the dual test cartridge, and then wait for the results. Assays that use other fluids, such as water samples essentially work in a similar manner. These devices are designed such that a typical layperson can perform the assays correctly with very little training. However, these assay systems generally suffer from low accuracy or require a number of preparative steps be performed that could compromise the test results and are thus not convenient.

In general, preparative steps of certain analytic assays involve configuring the assay system for a particular test or a particular type of test strip. If the assay is not configured properly, the results may be inaccurate if not misleading. Manual configuration may be prone to human error and adds another hurdle that the patient or end user has to pass before the sample may be tested.

Provided in certain embodiments herein is a (e.g., configurable) (e.g., diagnostic test) system comprising a test cartridge, an analyzer and a controller, and individual component parts thereof. In some embodiments, various methods and uses of the same are provided herein.

In some embodiments, provided herein is a dual-enclosure test cartridge (e.g., for implementing a diagnostic test). In some embodiments, the dual-enclosure test cartridge comprises a first component (e.g., a first body defining an inner enclosure) and a second component (e.g., a second body defining an outer enclosure). In some embodiments, the cartridge comprises a configurable parameter module, and a diagnostic test strip. In some embodiments, the cartridge comprises a configurable parameter module (e.g., within the second (e.g., outer) component). In some embodiments, the cartridge comprises a configurable parameter module and a diagnostic test strip. In specific embodiments, the cartridge comprises a first (e.g., inner) component comprises a first body and a diagnostic test strip. In some embodiments, the second (e.g., outer) component comprises a second body, wherein the second body at least partially enclosing the first (e.g., inner) component (e.g., with an aperture for applying a test material to the diagnostic test strip and/or viewing the test strip with an (e.g., optical) analyzer component). In some instances, use of a dual cartridge system facilitates reuse of a portion (e.g., outer enclosure) of the cartridge. In certain instances, such a cartridge allows for reducing waste by disposing of only a portion (e.g., inner enclosure) after use. In some instances, use of the outer cartridge facilitates uniformity of test cartridges across several assay platforms—in other words, a single outer enclosure configuration can be utilized with a variety of inner cartridge configuration and still be used in the same system, e.g., with the same controller and/or analyzer.

In some embodiments, provided herein is an (e.g., optical) analyzer comprising a diagnostic controller, the diagnostic controller communicatively coupled with the configurable parameter module of the dual-enclosure test cartridge. In certain specific embodiments, the diagnostic controller determines a configuration parameter for the dual-enclosure test cartridge based on an identification received from the configurable parameter module. In some specific embodiments, diagnostic controller configures the (e.g., optical) analyzer to perform a test based on the determined configuration parameter.

Provided herein is a configurable diagnostic test system comprising a dual-enclosure test cartridge (e.g., for implementing a diagnostic test) and an (e.g., optical) analyzer, the dual-enclosure test cartridge comprises an inner enclosure and an outer enclosure, the cartridge comprising: a configurable parameter module, and a diagnostic test strip; the (e.g., optical) analyzer comprising a diagnostic controller, the diagnostic controller communicatively coupled with the configurable parameter module of the dual-enclosure test cartridge, wherein the diagnostic controller (i) determines a configuration parameter for the dual-enclosure test cartridge based on an identification received from the configurable parameter module, and (ii) configures the (e.g., optical) analyzer to perform a test based on the determined configuration parameter. In some embodiments, the (e.g., optical) analyzer is multiplexing and is capable of running a plurality of tests. In some embodiments, each test of the plurality of tests is the same test or a different test. In some embodiments, the outer enclosure comprises a thermally conductive material. In some embodiments, the outer enclosure is textured and/or colored. In some embodiments, the outer enclosure comprises a (e.g., single) viewing aperture(s) to detect chemical processes and reactions, wherein the aperture(s) are covered or are open to the environment (e.g., wherein a cover is selected from the group consisting of a clear covering, an opaque covering, and any combinations thereof). In some embodiments, the outer enclosure comprises multiple viewing apertures to detect chemical processes and reactions (e.g., a result thereof), wherein the multiple viewing apertures are covered or are open to the environment (e.g., wherein the cover(s) are selected from the group consisting of a clear covering, an opaque covering, and any combinations thereof). In some embodiments, the outer enclosure comprises the configurable parameter module incorporated within or on the outer enclosure's body thereof. In some embodiments, the configurable parameter module comprises instructions (e.g., associated with the identification) for more than one diagnostic tests (e.g., to be conducted on the diagnostic test strip). In some embodiments, the analyzer further comprises a detector (e.g., an optical analyzer component capable of optically evaluating a diagnostic test strip (e.g., a portion thereof), such as through an aperture, or a cover thereof, of the outer enclosure). In some embodiments, the analyzer is a portable handheld analyzer. In some embodiments, the analyzer is a portable handheld optical analyzer. In some embodiments, the diagnostic test strip (or diagnostic test strips) are (collectively) configured to have one or more diagnostic test conducted thereon. In some embodiments, the inner enclosure comprises one or more diagnostic test strip(s) on the inner enclosure's body thereof. In some embodiments, the diagnostic test comprises a chemical reaction in which the chemical reaction produces a signal that is detectable by the (e.g., optical) analyzer (e.g., a detector thereof). In some embodiments, the diagnostic test comprises a chemical reaction, and the (e.g., optical) analyzer is capable of detecting or analyzing a signal produced by the chemical reaction. In some embodiments, the configurable parameter module stores parameter information associated with a first testing identification, wherein the stored parameter information is configured to be erased, removed, reused, or uploaded (e.g., thereby to be associated to a second testing identification). In some embodiments, the analyzer is powered by one or more batteries or a rechargeable battery pack. In some embodiments, the analyzer comprises a transmission component to electronically power the analyzer. In some embodiments, the analyzer comprises a transmission component, the transmission component configured to upload, or download new executable files (such as to facilitate communication between the (e.g., optical) analyzer and an external device). In some embodiments, the analyzer comprises wireless communication modules (e.g., configured to transmit to and receive information from an external device). In some embodiments, the analyzer comprises one or more detectors (e.g., configured to measure the chemical reaction of each test). In some embodiments, the analyzer is configured to (e.g., using a high-resolution assay) make up to 3 or more (e.g., 5 or more, or 10 or more) samples per second. In some embodiments, the analyzer is further configured to detect changes in signals from one or more apertures (e.g., which then can be used for computational analysis). In some embodiments, the diagnostic controller is configured with preset executable method files (e.g., arranged in tabular form). In some embodiments, wherein the analyzer is configured to (1) communicate with the configurable parameter module (e.g., after the cartridge is inserted into a receptacle thereof); (2) based on the communication from the configurable parameter module, access executable method files (e.g., stored on the analyzer); and (3) based on the executable method files, subject the test strip of the diagnostic test strip (e.g., which is inserted into a receptacle of the analyzer) to test conditions. In some embodiments, the (e.g., optical) analyzer comprises a dual sensor cartridge heating component (e.g., that is configured to control the heating source, such as while simultaneously sensing and controlling the cartridge temperature (e.g., using a two-level servo)). In some embodiments, the configurable diagnostic test system further comprises a graphic (e.g., color) (e.g., touch screen) display for user interface, result display, and/or user prompts, wherein the user prompts, in some cases, comprise instructions to a user when a predefined period of time has passed since a first sample is received by the dual-enclosure test cartridge. In some embodiments, the inner enclosure is removable and replaceable (e.g., with a second inner enclosure) from the dual-enclosure test cartridge.

Provided herein is a configurable diagnostic test system comprising a dual-enclosure test cartridge, the dual-enclosure test cartridge comprises a plurality of test zones, a port for receiving testing samples, and an identification module, and an analyzer that is capable of analyzing a plurality of tests, wherein the analyzer communicatively coupled with the identification module of the dual-enclosure test cartridge, and the analyzer (i) determines a particular test from the plurality of tests for the dual-enclosure test cartridge based on an identification received from identification module of the dual-enclosure test cartridge, and (ii) performs the determined test based on the determined identification. In some embodiments, the analyzer comprises a display screen, the display screen displays instruction based on a predetermined set of the rules associated with the particular test. In some embodiments, the analyzer comprises a display screen, the display screen displays test result associated with the particular test. In some embodiments, the dual-enclosure test cartridge comprises an outer enclosure and an inner enclosure. In some embodiments, the inner enclosure is removable from the dual-enclosure test cartridge, and may be replaced with a second inner enclosure. In some embodiments, the identification module of the dual-enclosure test cartridge is updated when the inner enclosure is replaced by a second inner enclosure. In some embodiments, the analyzer may communicate with the dual-enclosure test cartridge wirelessly. In some embodiments, the analyzer may receive the identification from the dual-enclosure test cartridge by placing the dual-enclosure in an opening of the analyzer. In some embodiments, the plurality of test zones includes a first test zone that measures the quantity of analyte in the received sample. In some embodiments, the plurality of test zones includes a second test zone that that capture one or more components of the received sample.

Provided herein is test cartridge comprising a first (e.g., inner) component, a second (e.g., outer) component and a configurable parameter module (preferably within the second (e.g., outer) component), the first (e.g., inner) component comprises a first body and a diagnostic test strip, wherein the first body at least partially enclosing the diagnostic test strip; the second (e.g., outer) component comprises a second body, wherein the second body at least partially enclosing the first (e.g., inner) component (e.g., with an aperture for applying a test material to the diagnostic test strip and/or viewing the test strip with an (e.g., optical) analyzer component).

In some embodiments, one or more part of a cartridge provided herein is recyclable. In some embodiments, the second (or outer) component is recyclable. In some embodiments, the first (or inner) component is recyclable. In some embodiments, the first (or inner) component and the second (or outer) component are recyclable. In some embodiments, the diagnostic test strip is for a single use.

The present disclosure provides, in certain embodiments, a cybernetic measurement device comprising control mechanisms such as temperature controls, timing mechanisms, electronic systems, informational communication systems, mechanical interlocks, the like, and/or any combinations thereof. The cybernetic measurement device may utilize chemistry and material science to produce a desired result.

In certain embodiments, the present disclosure provides systems and methods for configuring an analyzer that performs a diagnostic test using a foil pouched test cartridge. Any suitable test cartridge may be used and should not be limited herein. Any suitable analyzer capable of analyzing a sample may be used and should not be limited herein. In a non-limiting example, the analyzer may be an optical analyzer. In certain embodiments, the test cartridge comprises a parameter module that may store information and data regarding the test to be performed. The parameter module may also include parameters used to configure the analyzer to perform the associated diagnostic test. In another embodiment, the test cartridge may comprise a dual enclosure where the outer enclosure is disposed about the parameter module and an inner enclosure that may house specific chemical test for diagnosis. In another embodiment, the outer enclosure may be re-used. Any suitable enclosure and/or combinations of enclosure may be used and should not be limited herein. In an embodiment, a suitable enclosure may be capable of keeping out dust, fingers, chemicals in the air, physical materials, spurious electrical signals, optical signals, sonic signals, other signals, wind, bursts of heat, bursts of cold, other environmental conditions that may not be desired, the like, and/or any combinations thereof. A suitable enclosure may also be capable of retaining other components, heat, signals, materials, affluent, other factors and parameters produced by the internal process of the analyzer, the like, and/or any combinations thereof. In an embodiment, the enclosure may be capable of controlling the internal environment of the enclosure. In a non-limiting example, the enclosure may comprise an electronic control system capable of controlling and sensing light exposure and the temperature within the enclosure. Optionally, the parameter module may be configured with new test parameters. The test parameters may be determined in any suitable manner and should not be limited herein. In another embodiment, the inner enclosure may be formed from a thermally conductive material capable of absorbing heat from a heat source. The information module may include parameters that may be used to configure the diagnostic test to be performed by the testing strip. Any suitable parameter desired for a given test may be used and should not be limited herein.

The analyzer may comprise a diagnostic controller, a parameter module, a storage and a web based communication hardware module. The analyzer may comprise any suitable component necessary for configuration and testing and analyzing a sample. In an embodiment, the diagnostic controller may receive information from the test cartridge's parameter module. The diagnostic controller may then direct one of the other modules in the analyzer to perform a particular function. In a non-limiting example, the diagnostic controller may be configured to direct the association determination module to select specific algorithm files associated with the test cartridge. The test cartridge may be suitable for any diagnostic tests and should not be limited herein. In an embodiment, the diagnostic controller may receive a test cartridge identifier from the cartridge's parameter module. The diagnostic controller may then direct the parameter module to retrieve written algorithm codes from storage. The written algorithm codes may be in tabular form so one or more sets of codes may be selected to run a diagnostic test. The written algorithm codes may be in any suitable form and should not be limited herein. The analyzer may comprise any suitable storage unit capable of storing information, and should not be limited herein.

In an embodiment, the diagnostic controller may receive a test identifier or the test itself from the strip or the vial and may then transmit the received test identifier or the test to the diagnostic test module. The diagnostic test module may then configure the analyzer with the received test. Optionally, if the diagnostic test module receives a test identifier, the diagnostic test module retrieves from storage a test corresponding to the received test identifier and may then configure the analyzer with the retrieved test, such that a similar test may be performed on a sample.

In yet another embodiment, the diagnostic controller may direct the parameter module to configure the analyzer to perform a test using parameters corresponding to the received cartridge parameter module to perform a test using the retrieved parameters. The results of the test may then be transferred to storage. The results in storage may then be transmitted to a secondary device with internet and cellular service supporting networked capabilities using a web based communication hardware module. In a non-limiting example, the web based communication hardware module may be a wireless web based communication hardware module. Any suitable communication hardware module may be used and should not be limited herein.

In an embodiment, the analyzer may measure a parameter including, but not limited to, frequencies, the amount of photons, other signals or indications emitted by test cartridges, other signals or indications reflected from test cartridges, the like, and/or any combinations thereof. Optionally, the analyzer may measure a parameter of a test cartridge, wherein the test cartridge and the analyzer may be under different conditions and, wherein the analyzer may recreate the measured parameters. In a non-limiting example, the parameters that may be recreated by the analyzer may change as more measurements are collected. The parameters may be collected over a specified time period. In an embodiment, the analyzer may be capable of accepting a test cartridge and/or a plurality of test cartridges at a predefined location. In an embodiment, the test cartridge may be manually inputted into the analyzer. The analyzer may be capable of performing measurements by inducing a predetermined sequence of condition on a calibrated absorbency matrix material over a specified time period. The measurements produced by the analyzer may be used in conjunction with internal computational models and mechanisms, thereby producing a set of results in which the analyzer may be designed to produce.

In an embodiment, the analyzer may comprise a control sequencer that may be capable of the following: 1) reading an indicator from a test cartridge upon insertion into the analyzer thereby determining the type of absorbency matrix material present in the cartridge and its locations within the test cartridge; 2) based on predetermined instruction sets different temperatures for different time periods within the chamber; 3) collecting resulting data from the sensors within the chamber and may then record them as to value and time; 4) performing a predetermined analysis of the measurement results to produce defined resulting measurement values for each of the values being measured on each of the absorbency matrix materials; 5) providing status and results to a display module for user interface; the like; and/or any combinations thereof. Any suitable display module may be used and should not be limited herein. Suitable display modules may be capable of displaying information pertaining to an inserted cartridge, displaying information pertaining to the time and expected completion time of the measurement, displaying results indicating relevant measured values as they become available from the controller, the like, and/or any combinations thereof. The analyzer may further comprise a power source. Any suitable power source may be used including, but not limited to, an internal power source, and external power source, a wall plug, a battery, the like, and/or any combinations thereof. The power source may be capable of providing power to all of the components within the system.

In an embodiment, the test cartridge may be an independent device from the analyzer that may comprise the absorbency matrix material. The absorbency matrix material may comprise molecules that may be located at different locations and may be configured to interact with fluid samples. As the absorbency matrix material interacts with the fluid samples, the test cartridge may emit or reflect different parameters at different temperatures and/or under different rates of temperature change. In certain embodiments, the test cartridge or a plurality of test cartridges may be disposed within one or more defined locations within the analyzer. Optionally, the test cartridge may comprise indicators that may be communicated to the analyzer thereby conveying desired information including, but not limited to, specific parameters, properties, provenance information of the test cartridge, provenance information of the absorbency matrix material, the like, and/or any combinations thereof.

The test cartridge may comprise any suitable substrate for testing and may not be limited herein. In an embodiment, the testing substrate may comprise a piece of filter paper cut to shape and impressed with iodine and a blue dye. Optionally, the testing substrate may be at least partially encapsulated by a structure capable of comprising the absorbency matrix material. In an embodiment, the structure may be formed of any suitable material including, but not limited to, a metal, a plastic, the like, and/or any combinations thereof. Optionally, the test cartridge may comprise an identification number or code. The identification number or code may be used to retrieve information regarding the test cartridge (e.g., what the absorbency matrix material is composed of).

In certain embodiments, information may be communicated between the analyzer and the test cartridge in any suitable manner including, but not limited to, a network-based analytical system. The network-based analytical system may comprise network-based transmissions, communication, storage, retrieval, computation, the like, and/or any combinations thereof. In certain embodiments, the analyzer may communicate with the network-based analytical system. The network-based analytical systems may provide the analyzer with a potential of sequences of conditions in which the analyzer may induce in different samples over a given time period. Optionally, the network-based analytical systems may provide the analyzer with internal computational mechanisms or methods of analysis thereby producing results with different properties that may be associated with different absorbency matrix materials. The produced results may be used to inform the network-based analytical system of conditions and results within, around, and/or associated with the analyzer and/or test cartridge. The produced results may be used to support future analysis, calibration changes, new methods, new sequences, historical trends, indications of maintenance conditions, the like, and/or any combinations thereof.

The systems or components of the present disclosure may be used in a wide variety of applications including in the analysis of any number of fluids about which analytic information is desired to be determined (e.g., brewed fluids, natural fluids (e.g., juice), biological fluids (e.g., in a medical setting), etc.). In some specific embodiments, systems or components thereof provided herein can be used in, by way of non-limiting example, at the cask for brewed fluids (e.g., wine, beer, spirits, and the like), testing grape juice in a vineyard, a doctor's office, home medical testing, testing within medical facilities, testing during space exploration, veterinary clinic, agricultural facilities, manufacturing processes, rapid testing in a law enforcement department, industrial facilities (e.g., testing water and soil), the like, and/or any combinations thereof.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates a base system and a test cartridge according to one embodiment of the invention.

FIG. 2 illustrates an expanded view of the dual design test cartridge with outer enclosure and an inner enclosure where a test strips are enclosed according to one embodiment of the invention.

FIG. 3 illustrates an embodiment of a dual design test cartridge comprising of multiple open apertures corresponding to specific testing zones and an area for a parameter module according to one embodiment of the invention.

FIG. 4 is an embodiment of a block diagram that illustrates a computing device in the base system according to one embodiment of the invention.

FIG. 5 is an embodiment of a flow chart that illustrates the dynamic configuration of base system according to one embodiment of the invention.

### DETAILED DESCRIPTION

All publications, patents and patent applications cited herein, whether supra or infra, are hereby incorporated by reference in their entirety.

As used herein, the term “subject” may refer to mammals, non-mammals, chemical markers, industrial process chemical markers, environmental chemical markers, the like, or any combinations thereof. Non-limiting examples of mammals may include, but are not limited to, any member of the Mammalian class: humans, non-human primates including, but not limited to, chimpanzees, other apes, other monkey species, the like, and/or any combinations thereof; farm animals including, but not limited to, cattle, horses, sheep, goats, swine, the like, and/or any combinations thereof domestic animals including, but not limited to, rabbits, dogs, and cats, the like, and/or any combinations thereof laboratory animals including, but not limited to, rodents, such as rats, mice and guinea pigs, the like, and/or any combinations thereof. Examples of non-mammals may include, but are not limited to, birds, fish the like, and/or any combinations thereof. The term “subject” may not be limited to a particular age or gender. Suitable process biomarkers may include chemical makers such as derivatives of sugars, alcohols, proteins, nucleic acid such as DNA and RNA, amino acids, amino acid derivatives, lipids, lipid derivatives, cholesterol, oils, polymeric long chain polymers, polymeric short chain polymers, the like, and/or any combinations thereof. Suitable chemical markers may include, but are not limited to, acids, acid derivatives, bases, base derivatives, salts, salt derivatives, lead salts, arsenic salts, various simple salts that may be found in fermentation processes, industrial waste, mining soils, the like, and/or any combinations thereof

The term “antibody,” as used herein may refer to a polypeptide substantially encoded by an immunoglobulin gene or immunoglobulin genes, or fragments thereof, which specifically bind and recognize an analyte (antigen). “Antibody” as used herein may also refer to a polypeptide substantially encoded by an immunoglobulin gene or immunoglobulin genes, or fragments thereof, which specifically bind and recognize the antigen-specific binding region (idiotype) of antibodies produced by the host in response to exposure to trichomonas antigen(s). Non-limiting examples of antibodies may include, but are not limited to, polyclonal, monoclonal, chimeric, humanized, and single chain antibodies, the like, and/or any combinations thereof. Fragments of immunoglobulins may include, but are not limited to, Fab fragments and fragments produced by an expression library, including phage display. See, e.g., Paul, Fundamental Immunology, 3rd Ed., 1993, Raven Press, New York, for antibody structure and terminology.

The terms “specifically binds to” or “specifically immunoreactive with” may refer to a binding reaction, which may be determinative of the presence of the target analyte in the presence of a heterogeneous population of proteins and other biologics. Thus, under designated assay conditions, the specified binding moieties may bind preferentially to a particular target analyte and may not bind in a significant amount to other components present in a test sample. Specific binding to a target analyte under such conditions may require a binding moiety that may be selected for its specificity for a particular target analyte. Optionally, a variety of immunoassay formats may be used to select antibodies specifically immunoreactive with a particular antigen. In a non-limiting example, solid-phase ELISA immunoassays may be used to select monoclonal antibodies specifically immunoreactive with an analyte. See Harlow and Lane (1988) Antibodies, A Laboratory Manual, Cold Spring Harbor Publications, New York, for a description of immunoassay formats and conditions that may be used to determine specific immunoreactivity. In an embodiment, a specific or selective reaction may provide a signal to noise ratio of about at least twice background. In certain embodiments, a specific or selective reaction may provide a signal to noise ratio of about 10 to about 100 times background. The present disclosure may have any suitable signal to noise ratio and should not be limited herein.

As used herein, the terms “label” and “detectable label” may refer to any molecule capable of detection. Molecules capable of detection may include, but are not limited to, radioactive isotopes, fluoresces, chemical illuminescers, chromophores, enzymes, enzyme substrates, enzyme cofactors, enzyme inhibitors, chromophores, dyes, metal ions, metal sols, ligands, biotin, avidin, streptavidin, haptens, the like, and/or any combinations thereof. Any suitable molecule capable of detection may be used and should not be limited herein.

As used herein, an “absorbency matrix” may refer to a solid surface such as a plastic plate, magnetic bead, latex bead, microtiter plate well, glass plate, nylon, agarose, acrylamide, the like, and/or any combinations thereof. Any suitable absorbency matrix may be used and should not be limited herein.

“Specific” may refer to the binding of two molecules or a molecule and a complex of molecules, in which the specific recognition of one for the other and the formation of a stable complex as compared to substantially less recognition of other molecules and the lack of formation of stable complexes with such other molecules. In a non-limiting example, a specific binding may be antibody-antigen interactions, enzyme-substrate interactions, polynucleotide hybridizations and/or formation of duplexes, cellular receptor-ligand interactions, the like, and/or any combinations thereof.

The figures (Figs.) and the following description may relate to embodiments of the present disclosure by way of illustration only. It may be noted that from the following discussion, alternative embodiments of the structures and methods disclosed herein may be readily recognized as viable alternatives that may be employed without departing from the principles of what is claimed.

Reference may now be made in detail to several embodiments, examples of which are illustrated in the accompanying figures. It may be noted that wherever practicable similar or like reference numbers may be used in the figures and may indicate similar or like functionality.

The figures depict embodiments of the disclosed system (or method) for purposes of illustration only. One skilled in the art, along with the present disclosure, may recognize that alternative embodiments of the structures and methods illustrated herein may be employed without departing from the principles described herein.

As used herein any reference to “one embodiment,” “an embodiment,” or “some embodiments” means that a particular element, feature, structure, or characteristic described in connection with the embodiment may be included in at least one embodiment. The appearances of the phrase “in one embodiment” in various places in the specification are not necessarily all referring to the same embodiment.

Some embodiments may be described using the expression “coupled” and “connected” along with their derivatives. It may be understood that these terms may not be synonyms for each other. For example, some embodiments may be described using the term “connected” to indicate that two or more elements are in direct physical or electrical contact with each other. In another example, some embodiments may be described using the term “coupled” to indicate that two or more elements are in direct physical or electrical contact. The term “coupled,” however, may also mean that two or more elements are not in direct physical or electrical contact with each other, but yet still co-operate or interact with each other. The embodiments are not limited in this context.

Some embodiments may be described using “dual” design.

Optionally, some embodiments of the present disclosure may be further divided into logical modules. One of ordinary skill in the art, along with the present disclosure, may understand that these modules may be implemented in hardware, firmware and/or software. In one embodiment, the modules may be implemented in form of computer instructions that may be stored in a computer readable medium when executed by a processor. The processor may implement the functionality of the module. Additionally, one of ordinary skill in the art, along with the present disclosure, may recognize that a computer or another machine comprising instructions to implement the functionality of one or more logical modules may not be a general-purpose computer. Instead, the machine may be adapted to implement the functionality of a particular module. Optionally, the machine embodiment of the invention may physically transform the electrons representing the images from one state to another in order to attain the desired images.

As used herein, the terms “comprises,” “comprising,” “includes,” “including,” “has,” “having” or any other variation thereof, are intended to cover a non-exclusive inclusion. For example, a process, method, article or apparatus that comprises a list of elements may not necessarily be limited to only those elements but may include other elements not expressly listed or inherent to such process, method, article or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This may be done merely for convenience and to give a general sense of the disclosure. This description may be read to include one and/or at least one and the singular also includes the plural unless it is obvious that it may be meant otherwise.

The present disclosure may be directed towards a dual design test cartridge that may be used for performing qualitative and quantitative immuno and chemical assays. In certain embodiments, the dual design test cartridge may comprise at least two or more test zones. The test zones may be in fluidic communication with each other via capillary channels or absorbency matrix. The test cartridge may further comprise at least one or more ports, wherein the port may be capable of receiving a sample for analysis. Any suitable sample may be tested including, but not limited to, a liquid sample, a gas sample, a solid sample, the like, or any combinations thereof. Once the port receives a sample, the sample may then advance into the one or more test zone via capillary channels or absorbency matrix. A first test zone may measure the total analyte in the sample, thereby providing a standard analysis. The second test zone may be a reaction well, wherein individual components of the sample may be identified.

In certain embodiments, the dual test cartridge may be moved within a given proximity to an analyzer, such that the analyzer may then retrieve the desired information from the dual test cartridge. Upon retrieval of the desired information, the analyzer may then be configured to perform a diagnostic test using the particular dual test cartridge.

Optionally, the dual test cartridge may be placed within an analyzer that may detect the individual components of the sample and/or the total analyte in the sample. The analyzer may include a display system capable of displaying the results of the analysis. In certain embodiments, the display system may also be capable of providing instructions during the operation of the assay.

In a non-limiting example, analyzer may be configured to determine the percent total of hemoglobin A1 c (HbA1c) in human red blood cells. A sample of blood from a subject may be deposited in the port. The sample may be moved into two test zones via the capillary channels. In certain embodiments, the first test zone may comprise a reagent that lyses the cells thereby releasing the hemoglobin from the red blood cells. The concentration of hemoglobin in the first test zone may then be measured using infrared or ultraviolet measuring techniques and/or devices. In certain embodiments, the first test zone may comprise a lysate, a known amount of an antibody specific for HbA1c, and a magnetic stirrer. When blood moves into the first test zone, the magnet may mix the liquids test zone. The lysate may lyse the cells and the antibody may then bind to hemoglobin A1 c. After a specified period of time, the display may then instruct the operator to add a diluent to the first test zone. The diluent may push the liquid in the first test zone through another capillary channel towards a second test zone. In certain embodiments, the second test zone may comprise one or more capture zones. In a non-limiting embodiment, each capture zone may be split into two or more sections. A first section of the capture zone may comprise immobilized on them antigens that may bind to the bound antibody complex only. A second section of the capture zone may comprise other antigens that may bind to the antibodies. The antigens in the first section of the capture zone may capture the antibody-HbA1c complex, and the antigens in the second section of the capture zone may capture the antibodies. A detection system may be used to detect the antibodies bound in the first and the second sections of the capture zone. The ratio and/or the sum of the two zones may be used to quantify the amount of HbA1c present in the sample. The ratio of the first section to the total hemoglobin from the first test zone may provide the percentage of HbA1c in the blood sample. The results may be displayed on the display system in any suitable manner.

In another non-limiting example, the analyzer may be configured to determine the percent of fructose and glucose present in grapes. A sample of grape juice may be disposed in a sample port. The sample may then move to a first test zone via an absorbency matrix. The first test zone may comprise impregnated enzymes and indicators specific to fructose forms a colorimetric change. The sample may also move to a second test zone, wherein the second test zone may comprise enzymes and indicators specific to glucose and forms a colorimetric change. The analyzers using the test cartridge identification may quantify the percent of fructose and glucose in the sample. The results may then be displayed on the display system in any suitable manner.

In another non-limiting example, dual test cartridges (FIG. 2) may comprise an outer enclosure and inner enclosure. In certain embodiments, the outer enclosure and the inner enclosure may be formed from different injection molded resins or machinable material. Optionally, the outer enclosure and the inner enclosure may be formed from the same injection molded resin or machinable material. In certain embodiments, the outer enclosure may be colored to identify which test is being performed. Optionally, the inner enclosure may be made of a thermally conductive material such as a resin or metal. Any suitable thermally conductive material may be used and should not be limited herein. The dual test cartridge may be brought or moved within a given proximity to an analyzer, such that the analyzer may retrieve the desired information from the dual test cartridge. In certain embodiments, it may be advantageous to alter the temperature of the inner enclosure before testing a sample. A heating module may be disposed within the analyzer and may be triggered to provide enough energy such that the temperature of the inner enclosure may be adjusted to a predetermined temperature. A heating module may be any suitable heating element capable of providing enough energy (e.g., heat) to alter the temperature of the inner enclosure. The desired temperature of the inner enclosure may be predetermined by a user and/or the analyzer based on a number of parameters including, but not limited to, the sample to be tested, the analyte used, the desired measurement and/or analysis of the sample, the like, and/or any combinations thereof. Once the inner enclosure reaches the desired temperature, the sample may be added to the port. The sample may then be tested.

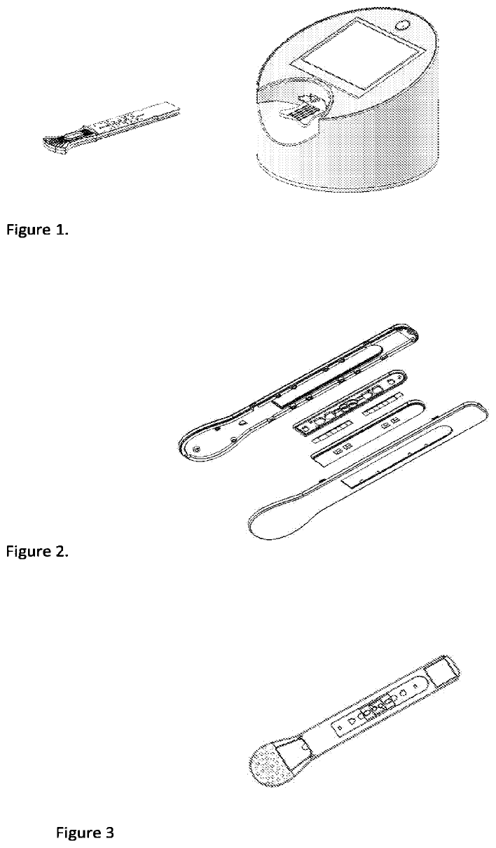

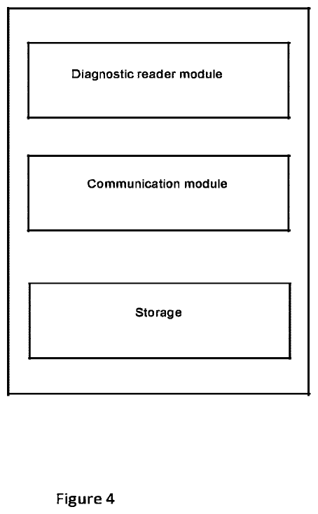

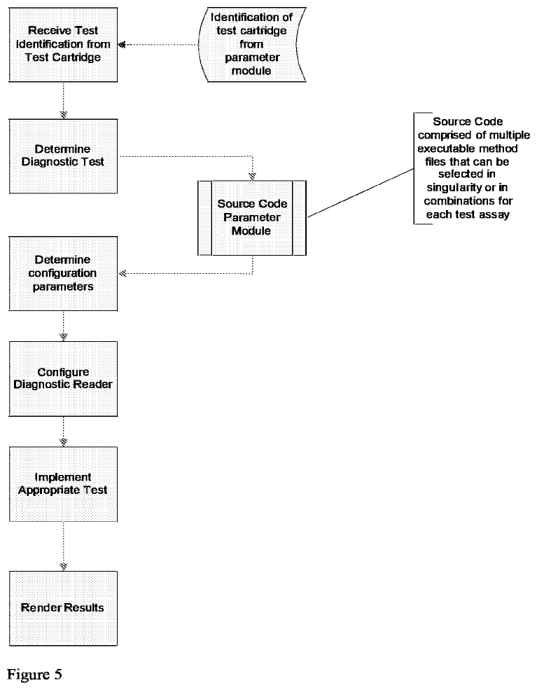

Referring to FIG. 1, illustrates an embodiment of a dual test cartridge and analyzer. FIG. 2 and FIG. 3 illustrate an embodiment of a dual test cartridge. FIG. 4 illustrates an embodiment of an analyzer.

The dual test cartridge further comprises a parameter module for a test assay. This test cartridge information module (e.g., a configurable parameter module) may be any module capable of storing vial information that may be retrieved at a later time by a reader. In a non-limiting example, the test cartridge information module may be an integrated circuit capable of storing and transmitting information through a wired or wireless connection, a radio frequency identification (RFID) tag, a visual tag with encoded information such as, a color-coded tag, a bar code, the like, and/or any combinations thereof. Information may be stored and transmitted through any suitable communication mode and should not be limited herein. In another embodiment, the test cartridge information may include the test and/or an identification of the test to be performed by the analyzer. In another non-limiting example, the test cartridge information may include configuration parameters for the analyzer. These parameters may be specific to various tests performed. The parameters and the tests may be described in greater detail below. In one embodiment, the parameters may be communicated or received by an analyzer though RFID tag, a visual tag with encoded information such as, a color-coded tag, a bar code, the like, and/or any combinations thereof. The analyzer may be configured with suitable components that may read the above-mentioned tags and thus configure itself based on the received information (e.g., parameters).

Optionally, the test cartridge may comprise test cartridge information module that may be capable of storing information about the test cartridge. The test cartridge information module may be any module capable of storing test cartridge information for later retrieval by a reader. Any suitable reader may be used to retrieve information and should not be limited herein. In a non-limiting example, the test cartridge information module may be an integrated circuit capable of storing and transmitting information through a wired or wireless connection, an RFID tag, or a visual tag with encoded such as a color-coded tag, a bar code, the like, and/or any combinations thereof. In another embodiment, the test cartridges outer enclosure may be re-used and/or recycled where the inner cartridge may be removed. The recycled outer enclosure may then be uploaded with new test cartridge information and a new unused inner cartridge enclosure.

In an embodiment, the analyzer may retrieve configuration information from a test cartridge. The analyzer may retrieve information from the test cartridge when the analyzer and the test cartridge may be brought in proximity to one another. As used herein, proximity may refer to a predetermined distance that the analyzer and the test cartridge must be. Proximity may be defined by a predetermined area. The predetermined area may be any area size and/or shape and should not be limited herein. One of ordinary skill in the art, along with the present disclosure, may be able to determine the appropriate proximity requirements for a given application. The proximity requirements may vary depending on the means used to store the information on test cartridge and any other parameter that may affect the transfer of information. In a non-limiting example, the information may be stored in the form of bar codes in which, the analyzer may use a scanner to read the information when the test cartridge may be within a few inches or a few feet.

The information may be retrieved in any suitable manner and should not be limited herein. The analyzer may then use the retrieved information to then configure itself with minimal input from the user. Accordingly, the configuration of the analyzer may increase test accuracy and reduce possibility of test errors, by minimizing the amount of possible human error.

FIG. 2 illustrates and embodiment of a test cartridge. The test cartridge may be formed by coupling and/or connecting two or more solid supports with grooves present in at least one of the supports. The solid support may be of any suitable size or shape. In an embodiment, the solid support may have any suitable shape including, but not limited to, rectangular, circular, oval, hexagonal, octagonal, heptagonal, the like, and/or any combinations thereof. In an embodiment, when the solid supports are joined together, they may comprise a solid outer surface and a hollow inner cavity. The support may be formed from a suitable material that may be selected for its properties, such as good thermal conductivity, clarity for optical transmission, mechanical properties for easy welding, surface properties that allow for uniform coating and stability of reagent, neutrality to the liquid medium to prevent interference with the assay, the like, and/or any combinations thereof. Suitable materials may include plastics and/or resins comprising high free surface energies and low water absorption. Examples of suitable materials may include, but are not limited to, PETG, polyester, polyethylene terephthalate, polycarbonate, polyvinyl chloride, polystyrene, acrylonitrile styrene copolymer, acrylonitrile-butadiene-styrene, the like, and/or any combinations thereof. In certain embodiments, the material may further comprise a thermal conducting agent. Any suitable thermal conducting agent capable of increasing the rate of heat transfer from a heat source to the support may be used. Suitable conducting agents may include, but are not limited to, carbon graphite, the like, and/or any combinations thereof. In certain embodiments, the solid support may be a hydrophobic plastic, which may be treated by art-known methods to render the surfaces hydrophilic, such as by plasma etching or by corona treatment. Alternatively, a commercially available molded solid support may be used in the practice of the disclosure.

What is claimed is:

1. A configurable diagnostic test system comprising:
a dual-enclosure test cartridge (e.g., for implementing a diagnostic test), the dual-enclosure test cartridge comprises an inner enclosure and an outer enclosure, the cartridge comprising:
a configurable parameter module, and
a diagnostic test strip;
an (e.g., optical) analyzer comprising a diagnostic controller, the diagnostic controller communicatively coupled with the configurable parameter module of the dual-enclosure test cartridge,
wherein the diagnostic controller
(i) determines a configuration parameter for the dual-enclosure test cartridge based on an identification received from the configurable parameter module, and
(ii) configures the (e.g., optical) analyzer to perform a test based on the determined configuration parameter.

2. The configurable diagnostic test system of claim 1, wherein the (e.g., optical) analyzer is multiplexing and is capable of running a plurality of tests.

3. The configurable diagnostic test system of claim 2, wherein each test of the plurality of tests is the same test or a different test.

4. The configurable diagnostic test system of any one of the preceding claims, wherein the outer enclosure comprises a thermally conductive material.

5. The configurable diagnostic test system of any one of the preceding claims, wherein the outer enclosure is textured and/or colored.

6. The configurable diagnostic test system of any one of the preceding claims, wherein the outer enclosure comprises a (e.g., single) viewing aperture(s) to detect chemical processes and reactions, wherein the aperture(s) are covered or are open to the environment (e.g., wherein a cover is selected from the group consisting of a clear covering, an opaque covering, and any combinations thereof).

7. The configurable diagnostic test system of any one of the preceding claims, wherein the outer enclosure comprises multiple viewing apertures to detect chemical processes and reactions (e.g., a result thereof), wherein the multiple viewing apertures are covered or are open to the environment (e.g., wherein the cover(s) are selected from the group consisting of a clear covering, an opaque covering, and any combinations thereof).

8. The configurable diagnostic test system of any one of the preceding claims, wherein the outer enclosure comprises the configurable parameter module incorporated within or on the outer enclosure's body thereof

9. The configurable diagnostic test system of any one of the preceding claims, wherein the configurable parameter module comprises instructions (e.g., associated with the identification) for more than one diagnostic tests (e.g., to be conducted on the diagnostic test strip).

10. The configurable diagnostic test system of any one of the preceding claims, wherein the analyzer further comprises a detector (e.g., an optical analyzer component capable of optically evaluating a diagnostic test strip (e.g., a portion thereof), such as through an aperture, or a cover thereof, of the outer enclosure).

11. The configurable diagnostic test system of any one of the preceding claims, wherein the analyzer is a portable handheld analyzer.

12. The configurable diagnostic test system of any one of the preceding claims, wherein the analyzer is a portable handheld optical analyzer.

13. The configurable diagnostic test system of any one of the preceding claims, wherein the diagnostic test strip (or diagnostic test strips) are (collectively) configured to have one or more diagnostic test conducted thereon.

14. The configurable diagnostic test system of any one of the preceding claims, wherein the inner enclosure comprises one or more diagnostic test strip(s) on the inner enclosure's body thereof

15. The configurable diagnostic test system of any one of the preceding claims, wherein the diagnostic test comprises a chemical reaction in which the chemical reaction produces a signal that is detectable by the (e.g., optical) analyzer (e.g., a detector thereof).

16. The configurable diagnostic test system of any one of the preceding claims, wherein the diagnostic test comprises a chemical reaction, and the (e.g., optical) analyzer is capable of detecting or analyzing a signal produced by the chemical reaction.

17. The configurable diagnostic test system of any one of the preceding claims, wherein the configurable parameter module stores parameter information associated with a first testing identification.

18. The configurable diagnostic test system of claim 17, wherein the stored parameter information is configured to be erased, removed, reused, or uploaded (e.g., thereby to be associated to a second testing identification).

19. The configurable diagnostic test system of any one of the preceding claims, wherein the analyzer is powered by one or more batteries or a rechargeable battery pack.

20. The configurable diagnostic test system of any one of the preceding claims, wherein the analyzer comprises a transmission component to electronically power the analyzer.

21. The configurable diagnostic test system of any one of the preceding claims, wherein the analyzer comprises a transmission component, the transmission component configured to upload, or download new executable files (such as to facilitate communication between the (e.g., optical) analyzer and an external device).

22. The configurable diagnostic test system of any one of the preceding claims, wherein the analyzer comprises wireless communication modules (e.g., configured to transmit to and receive information from an external device).

23. The configurable diagnostic test system of any one of the preceding claims, wherein the analyzer comprises one or more detectors (e.g., configured to measure the chemical reaction of each test).

24. The configurable diagnostic test system of any one of the preceding claims, wherein the analyzer is configured to (e.g., using a high-resolution assay) make up to 3 or more (e.g., 5 or more, or 10 or more) samples per second.

25. The configurable diagnostic test of claim 24, wherein the analyzer is further configured to detect changes in signals from one or more apertures (e.g., which then can be used for computational analysis).

26. The configurable diagnostic test system of any one of the preceding claims, wherein the diagnostic controller is configured with preset executable method files (e.g., arranged in tabular form).

27. The configurable diagnostic test system of any one of the preceding claims, wherein the analyzer is configured to (1) communicate with the configurable parameter module (e.g., after the cartridge is inserted into a receptacle thereof); (2) based on the communication from the configurable parameter module, access executable method files (e.g., stored on the analyzer); and (3) based on the executable method files, subject the test strip of the diagnostic test strip (e.g., which is inserted into a receptacle of the analyzer) to test conditions.

28. The configurable diagnostic test system of any one of the preceding claims, wherein the (e.g., optical) analyzer comprises a dual sensor cartridge heating component (e.g., that is configured to control the heating source, such as while simultaneously sensing and controlling the cartridge temperature (e.g., using a two-level servo)).

29. The configurable diagnostic test system of any one of the preceding claims, further comprising a graphic (e.g., color) (e.g., touch screen) display for user interface, result display, and/or user prompts.

30. The configurable diagnostic test system of claim 29, wherein the user prompts comprise instructions to a user when a predefined period of time has passed since a first sample is received by the dual-enclosure test cartridge.

31. The configurable diagnostic test system of the preceding claims, wherein the inner enclosure is removable and replaceable (e.g., with a second inner enclosure) from the dual-enclosure test cartridge.

32. A configurable diagnostic test system comprising:
a dual-enclosure test cartridge, the dual-enclosure test cartridge comprises a plurality of test zones, a port for receiving testing samples, and an identification module, and
an analyzer that is capable of analyzing a plurality of tests, wherein the analyzer communicatively coupled with the identification module of the dual-enclosure test cartridge, and the analyzer (i) determines a particular test from the plurality of tests for the dual-enclosure test cartridge based on an identification received from identification module of the dual-enclosure test cartridge, and (ii) perform the determined test based on the determined identification.

33. The configurable diagnostic test system of any one of the preceding claims, the analyzer comprises a display screen, the display screen displays instruction based on a predetermined set of the rules associated with the particular test.

34. The configurable diagnostic test system of any one of the preceding claims the analyzer comprises a display screen, the display screen displays test result associated with the particular test.

35. The configurable diagnostic test system of any one of the preceding claims wherein the dual-enclosure test cartridge comprises an outer enclosure and an inner enclosure.

36. The configurable diagnostic test system of claim 35, wherein the inner enclosure is removable from the dual-enclosure test cartridge, and may be replaced with a second inner enclosure.

37. The configurable diagnostic test system of claim 36, wherein the identification module of the dual-enclosure test cartridge is updated when the inner enclosure is replaced by a second inner enclosure.

38. The configurable diagnostic test system of any one of the preceding claims, wherein the analyzer may communicate with the dual-enclosure test cartridge wirelessly.

39. The configurable diagnostic test system of any one of the preceding claims, the analyzer may receive the identification from the dual-enclosure test cartridge by placing the dual-enclosure in an opening of the analyzer.

40. The configurable diagnostic test system of any one of the preceding claims, wherein the plurality of test zones includes a first test zone that measures the quantity of analyte in the received sample.

41. The configurable diagnostic test system of any one of the preceding claims, wherein the plurality of test zones includes a second test zone that that capture one or more components of the received sample.

42. A test cartridge comprising:
a first (e.g., inner) component comprises a first body and a diagnostic test strip, wherein the first body at least partially enclosing the diagnostic test strip;
a second (e.g., outer) component comprises a second body, wherein the second body at least partially enclosing the first (e.g., inner) component (e.g., with an aperture for applying a test material to the diagnostic test strip and/or viewing the test strip with an (e.g., optical) analyzer component), and
a configurable parameter module (preferably within the second (e.g., outer) component).

43. The test cartridge of claim 42, wherein the second component is recyclable.

44. The test cartridge of claim 42, wherein the first component is recyclable.

45. The test cartridge of claim 42, wherein the first component and the second component are recyclable.

46. The test cartridge of claim 42, wherein the diagnostic test strip is for a single use.

47. The configurable diagnostic test system of claims 1-41, wherein the dual-enclosure test cartridge comprises the test cartridge of claim 42.
